(19) United States
(12) Patent Application Publication
(10) Pub. No.: US 2006/0216315 A1
(43) Pub. Date: Sep. 28, 2006

(54) Cockroach allergen gene expression and delivery systems and uses

(76) Inventor: Tai June Yoo, Memphis, TN (US)

Correspondence Address: FOLEY & LARDNER LLP, P.O. BOX 80278, SAN DIEGO, CA, 92138-0278, US

(21) Appl. No.: 11/384,041

(22) Filed: Mar. 16, 2006

Related U.S. Application Data
(60) Provisional application No. 60/663,033, filed on Mar. 16, 2005.

(51) Int. Cl. A61K 48/00; A61K 39/35

(52) U.S. Cl. 424/275.1; 514/44

(57) ABSTRACT

The invention relates to compositions and methods for managing cockroach allergies in mammals. In particular aspects, the invention relates to the administration to a mammal of nucleic acids that encode a cockroach allergen. The compositions are prepared and administered in such a manner that the cockroach allergen coding sequence is expressed in the mammal to which the composition is administered. The compositions include expression systems, delivery systems, and certain cockroach allergens coding sequences.

### RELATED APPLICATIONS

The present application claims the benefit under 35 U.S.C. § 119(e) of U.S. Application Ser. No. 60/663,033 filed Mar. 16, 2005, the entire contents of which including all figures and tables are incorporated herein by reference.

### FIELD OF THE INVENTION

The invention relates to compositions and methods for managing allergies in mammals. In particular, the invention relates to cockroach allergens.

### BACKGROUND OF THE INVENTION

The incidence and prevalence of allergic diseases are increasing throughout the world, and their impact on quality of life of children and adults is significant. Theories put forth that try to explain this phenomenon include increasing air pollution, increased time spent indoors by children and adults, and decreased rates of infection seen in developed countries—the “hygiene” hypothesis (Liu, J Allergy Clin. Immunol. 109:379-92 (2002)). Cockroach allergen has been recognized as a major indoor allergen—initially in crowded urban settings (Garcia, et al., Ann. Allergy 72:203-8 (1994)), and now recognized in suburban and rural settings as well (Garcia, et al., Ann. Allergy 72:203-8 (1994); Hulett, et al., Ann Allergy 42:160-65 (1979); and Twarog, et al., J Allergy Clin. Immunol. 59:154-160 (1976)). Diseases such as allergic asthma, rhinitis, and atopic dermatitis are characterized by elevated levels of serum IgE. Inner city asthmatics often show elevated levels of cockroach specific IgE, with rates up to 60%. Cockroach species most commonly found in the United States is the American cockroach, Periplaneta americana, and the German cockroach, Blatella germanica. Several cockroach allergens associated with the American and German cockroach species have been idenfified including Bla g 1, Bla g 2, Bla g 4, Bla g 5, Bla g 6, Per a 1, Per a 2 and Per a 3 (Chapman, et al., “Allergy and Allergic Disease,” Ed. A. B. Kay, Blackwell Scientific Publ., Oxford, UK 1996).

The treatment of allergic diseases consists of three complimentary measures: avoidance, pharmacotherapy, and specific immunotherapy. Avoidance of cockroach is difficult. Infestation occurs in large apartment buildings, office buildings, and homes, and eradication is difficult, if not impossible. Pharmacotherapy consists of using antihistamines and inhaled corticosteroids, long acting β₂ agonists and leukotriene receptor antagonists to decrease nasal allergy symptoms and to a mild degree, eye allergy symptoms. Treatment of chronic allergic conditions is made more difficult by the issue of compliance. The third measure, immunotherapy, offers a great measure of relief to many individuals whose symptoms are not well controlled by avoidance and medications. Traditional immunotherapy includes a long build-up phase, which may take as long as 6 to 12 months, and a prolonged immunization course, up to 5 years or more. The risk of a systemic reaction prevents many of the most needy patients (severe allergic asthmatics) from receiving this beneficial therapy. Many patients, after “finishing” a successful course of immunotherapy, have a recurrence of their symptoms that requires a reinstitution of allergy desensitization.

U.S. Pat. No. 5,869,288 (the '288 patent) by Chapman et al. describes potential immunogenic cockroach allergens. The '288 patent at column 2, lines 35-50 states “[t]he information obtained also permits the structural modification of the molecules, and alteration of specific amino acid residues in proteins, to identify specific amino acid residues recognized by IgE antibodies. The sequence information also allows the practitioner to design short peptides which can be chemically synthesized and tested for their ability to induce T-cell response in allergic patients. These responses control IgE antibody production, and the identification of appropriate peptides is a key step in developing a vaccine. Similarly, modalities for addressing reactivity by alternating the three-dimensional structure of each allergen, and recombinant expression for use in allergy diagnosis and in treatment, is made possible by this information;” and at column 10, lines 28-35 states “[t]he present studies will make it possible to develop new immunotherapeutic strategies for CR allergy, including T cell based vaccines, and will also facilitate further analysis of the molecular events that mediate chronic inflammatory responses in CR allergic patients with asthma. One important step in their progress is the expression of recombinant allergens, discussed above.”

### SUMMARY OF THE INVENTION

The invention relates to compositions and methods for managing cockroach allergies in mammals. In particular aspects, the invention relates to DNA vaccines useful for treatment of cockroach allergies in mammals.

In one aspect, the invention provides a method of treating or prophylaxis of an allergy in a mammal which includes administering to the mammal a polynucleotide including a sequence encoding a cockroach allergen or fragment thereof. In a related aspect, the invention provides a method for generating an immune response in a mammal which includes administering to the mammal a polynucleotide including a sequence encoding a cockroach allergen or fragment thereof. In another aspect the invention provides a method for detecting sensitivity to a cockroach allergen in an individual, wherein the method includes administering a polynucleotide including a sequence encoding a cockroach allergen or a fragment thereof to the individual and determining an allergic response. The invention also provides compositions that are suitable for administration to a mammal, preferably to a human. In one aspect, the invention provides a composition that includes a pharmaceutically acceptable carrier and a polynucleotide including a sequence encoding a cockroach allergen or fragment thereof. In a related aspect, the invention provides a method of preparing a composition for expression of a cockroach allergen or fragment thereof, in a mammal which includes preparing a polynucleotide including a promoter enhancer transcriptionally linked to a sequence encoding a cockroach allergen or a fragment thereof; preparing a transfection facilitating material; and combining the transfection facilitating material with the polynucleotide. In other aspects the invention provides a pharmaceutical kit which includes a container suitable for holding a pharmaceutical for administration to a human, a polynucleotide including a sequence encoding a cockroach allergen, a pharmaceutically acceptable carrier, and a label affixed to the container or a package insert. In yet other aspects, the invention provides a polypeptide including a sequence homologous to a cockroach allergen or a fragment thereof.

In preferred embodiments of the above aspects of the invention, the cockroach allergen of the methods and compositions is homologous to a cockroach allergen selected from the group consisting of Bla g 1, Bla g 2, Bla g 4, Bla g 5, Bla g 6, Per a 1, Per a 3, and Per a 7. In other preferred embodiments the polynucleotide of the methods and compositions of the invention includes a sequence encoding a fragment of a cockroach allergen with an amino acid sequence homologous to a sequence of a cockroach allergen selected from the group consisting of Bla g 1 (SEQ ID NO:9), Bla g 2 (SEQ ID NO: 10), Bla g 4 (SEQ ID NO:11), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO:16); preferably the fragment of a cockroach allergen includes an amino acid sequence that is at least 70% homologous to a sequence of a cockroach allergen selected from the group consisting of Bla g 1 (SEQ ID NO:9), Bla g 2 (SEQ ID NO:10), Bla g 4 (SEQ ID NO: 1), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO:16); preferably the fragment of a cockroach allergen includes at least 25 amino acids homologous to a sequence of a cockroach allergen selected from the group consisting of Bla g 1 (SEQ ID NO:9), Bla g 2 (SEQ ID NO:10), Bla g 4 (SEQ ID NO: 1), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO: 16). In other preferred embodiments the polynucleotide of the methods and compositions of the invention includes a nucleotide sequence of at least 100 contiguous nucleotides that are homologous to a nucleotide sequence selected from the group consisting of Bla g 1 (SEQ ID NO:1), Bla g 2 (SEQ ID NO:2), Bla g 4 (SEQ ID NO:3), Bla g 5 (SEQ ID NO:4), Bla g 6 (SEQ ID NO:5), Per a 1 (SEQ ID NO:6), Per a 3 (SEQ ID NO:7), and Per a 7 (SEQ ID NO:8); preferably the polynucleotide includes a nucleotide sequence of at least 100 contiguous nucleotides that are at least 70% identical to a nucleotide sequence selected from the group consisting of Bla g 1 (SEQ ID NO:1), Bla g 2 (SEQ ID NO:2), Bla g 4 (SEQ ID NO:3), Bla g 5 (SEQ ID NO:4), Bla g 6 (SEQ ID NO:5), Per a 1 (SEQ ID NO:6), Per a 3 (SEQ ID NO:7), and Per a 7 (SEQ ID NO:8).

In other preferred embodiments, the polynucleotides of the invention may be administered such that a combination two or more different cockroach allergens or fragments thereof are expressed in a mammal. This may be achieved either by administering two or more different polynucleotides that include a sequence encoding different cockroach allergens or fragments thereof, and/or by administering a polynucleotide that includes two or more sequences encoding different cockroach allergens or fragments thereof. In particularly preferred embodiments the combination includes a cockroach allergen with an amino acid sequence homologous to Bla g 1 or a fragment thereof and a cockroach allergen with an amino acid sequence homologous to Bla g 2 or a fragment thereof.

In other preferred embodiments, the polynucleotide of the methods and compositions of the invention is circular DNA; preferably the polynucleotide is a plasmid; preferably the polynucleotide includes a promoter/enhancer transcriptionally linked to the sequence encoding a cockroach allergen; preferably the promoter is suitable for expression in eukaryotic cells; in some preferable embodiments the polynucleotide is a viral vector.

In certain preferred embodiments of the methods of the invention, the polynucleotide including a sequence encoding a cockroach allergen or fragment thereof is administered to a mammal; more preferably the mammal is a human; preferably the polynucleotide including a sequence encoding a cockroach allergen or fragment thereof is administered with a transfection facilitating material; preferably the transfection facilitating material includes a lipid; preferably the polynucleotide is administered in a pharmaceutically acceptable carrier; in certain preferred embodiments the polynucleotide is administered by viral transduction; preferably the polynucleotide is administered by gene gun; preferably the polynucleotide is administered by inhalation; or preferably the polynucleotide is administered by injection, or preferably subcutaneous injection or more preferably intramuscular injection.

In certain preferred embodiments, the composition of the invention includes a polynucleotide including a sequence encoding a cockroach allergen or fragment thereof; preferably the composition includes a pharmaceutically acceptable carrier; preferably the composition includes a transfection facilitating material, preferably the transfection facilitating material includes a lipid; preferably the composition is administered with an adjuvant; preferably the composition is suitable for injection into a mammal, preferably the mammal is a human; preferably the composition is suitable for inhalation by a mammal, preferably the mammal is a human; preferably the composition is enclosed in a pharmaceutically acceptable carrier, preferably the pharmaceutically acceptable carrier has a label indicating the contents therein and a statement regarding administration of the polynucleotide; preferably the composition includes a package insert, preferably the package insert includes statements regarding the contents of the composition, more preferably dosing information.

As used herein, the term “plasmid” refers to circular DNA. In certain preferred embodiments of the invention, a plasmid is capable of replicating in prokaryotic cells and/or eukaryotic cells; more preferably a plasmid includes genetic elements arranged such that an inserted coding sequence can be transcribed in eukaryotic cells. Preferably, plasmids may be introduced into cells by transformation and can replicate autonomously in the cell.

The term “replication origin” “origin of replication” as used herein refers to a nucleotide sequence at which DNA synthesis for the purpose of replicating the nucleic acid sequence begins. This is generally termed an ORI site. Circular bacteria generally have a single ORI site, whereas there can be many ORI sites on each eukaryotic chromosome. This term includes replicons, which as used herein refers to a genetic element that behaves as an autonomous unit during DNA replication. In bacteria, the chromosome functions as a single replicon, whereas eukaryotic chromosomes contain hundreds of replicons in series.

As used herein, the term “expression” refers to the biological production of a product encoded by a coding sequence. In most cases a DNA sequence, including the coding sequence, is transcribed to form a messenger RNA (mRNA). Messenger RNA is translated to form a polypeptide product which has biological activity. However in some cases, an RNA product may have the relevant activity and would thus be regarded as a gene product. Expression may involve further processing steps of the transcription RNA product, such as splicing to remove introns, and/or post-translational processing of a polypeptide product.

The term “transcription unit” or “expression cassette” refers to a nucleotide sequence which contains at least one coding sequence along with sequence elements which direct the initiation and termination of transcription. A transcription unit may however include additional sequences, which may include sequences involved in post-transcriptional or post-translational processes.

As used herein, the term “coding region” or “coding sequence” refers to a nucleic acid sequence, its complement, or a part thereof, which encodes a particular gene product or a fragment thereof for which expression is desired, according to the normal base pairing and codon usage relationships. Coding sequences include exons in genomic DNA or immature primary RNA transcripts, which are joined together by the cell's biochemical machinery to provide a mature mRNA. The anti-sense strand is the complement of such a nucleic acid, and the encoding sequence can be deduced from there. The coding sequence is placed in relationship to transcriptional control elements and to translational initiation and termination codons so that a proper length transcript will be produced and will result in translation in the appropriate reading frame to produce a functional desired product.

The term “complement” “complementary” or “complementarity” as used herein with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). A complementary sequence can also be a sequence of RNA complementary to the DNA sequence or its complement sequence, and can also be a cDNA.

As used herein, the term “transcriptional control sequence” refers to a sequence which controls the rate of transcription of a transcriptionally linked coding region. The term can include elements such as promoters, operators, and enhancers. Preferably, the transcriptional control sequence will include at least one promoter sequence.

The term “allergy” is used herein as the term is known in the art and refers to the reaction of an immune system to a foreign substance.

As used herein, the term “pharmaceutically acceptable carrier” refers to a composition which is suitable for administration to a mammal, more preferably, to a human. Techniques for formulation and administration may be found, for example, in “Remington's Pharmaceutical Sciences,” (18th ed., Mack Publishing Co., Easton, Pa., 1990).

As used herein, the term “transcriptionally linked” refers to a system suitable for transcription, which will initiate under the direction of a control sequence and proceed through sequences which are transcriptionally linked with that control sequence. Preferably, no mutation is created in the resulting transcript which would alter the resulting translation product.

As used herein, the term “5′ untranslated region” or “5′ UTR” refers to a sequence located 3′ to promoter region and 5′ of the downstream coding region. Thus, such a sequence, while transcribed, is upstream (i.e. 5′) of the translation initiation codon and therefore is generally not translated into a portion of the polypeptide product.

As used herein, the term “3′ untranslated region/poly (A) signal” or “3′ UTR poly (A) signal” is a sequence located downstream (i.e., 3′) of the region encoding a cockroach allergen. As with the 5′ UTR, this region is generally transcribed but not translated. For expression in eukaryotic cells it is generally preferable to include a sequence which signals the addition of a poly-A tail. As with other synthetic genetic elements, a synthetic 3′ UTR/poly (A) signal has a sequence which differs from naturally-occurring, UTR elements.

As used herein, the term “Cytomegalovirus promoter/enhancer sequence” refers to a sequence from a cytomegalovirus which is functional in eukaryotic cells as a transcriptional promoter and an upstream enhancer sequence. The enhancer sequence allows transcription to occur at a higher frequency from the associated promoter.

For the plasmids described herein, one or more of a promoter, 5′ untranslated region (5′ UTR), 3′ UTR/poly (A) signal, and introns may be a synthetic sequence. In this context, the term “synthetic” refers to a sequence that is not provided directly by the sequence of a naturally occurring genetic element of that type but rather is an artificially created sequence (i.e., created by an individual by molecular biological methods). While one or more portions of such a synthetic sequence may be the same as portions of naturally occurring sequences, the full sequence over the specified genetic element is different from a naturally occurring genetic element of that type. The use of such synthetic genetic elements allows the functional characteristics of that element to be appropriately designed for the desired function.

As used herein, the term “treating” refers to the administration of an agent (for example a polynucleotide or polypeptide) to mammal. Although it is preferred that treating a condition such as an allergy will result in an improvement of the condition, the term treating as used herein does not indicate, imply, or require that the administration of the agent is at all successful in reducing or ameliorating symptoms associated with any particular condition. Indeed, treating an individual as contemplated herein may result in adverse side effects or even a worsening of the condition which the treatment was intended to improve.

As used herein, the term “prophylaxis” refers to a measure taken for the prevention of a disease or condition. As such the term does not indicate, imply, or require that the measure taken is at all successful in preventing the disease.

As used herein, the term “administration” or “administer” or “administering” refers to dispensing, applying, or tendering an agent (for example a polynucleotide or polypeptide) to a mammal. Administration may be performed using any of many methods known in the art.

As used herein, the term “including” has the same meaning as the term comprising.

As used herein, the term “cockroach allergen” refers to a polypeptide with an amino acid sequence that is homologous to a polypeptide naturally produced by a cockroach that causes an allergic reaction in certain mammals such as dermatitis, runny nose, allergic rhinitis, asthma, and anaphylaxis. Some cockroach allergens are well known in the art and include, but are not limited to Bla g 1 (GB Accession No. AF072219), Bla g 2 (GB Accession No. U28863), Bla g 4 (GB Accession No. U40767), Bla g 5 (GB Accession No. U92412), Bla g 7 (GB Accession No. AF260897), Per a 1 (GB Accession No. U69957), Per a 3 (GB Accession No. L40818), and Per a 7 (GB Accession No. AF106961); however it is understood that other cockroach allergens are also a subject of the invention, for example new cockroach allergens not yet identified. Preferably a cockroach allergen, or fragment thereof, of the invention has an amino acid sequence that is homologous to an amino acid sequence of a cockroach allergen as provided herein, i.e. SEQ ID NOs: 9-16. In certain preferred embodiments a fragment of a cockroach allergen has at least 25 amino acids, more preferably at least 50 amino acids, more preferably at least 150 amino acids, more preferably at least 200 amino acids, more preferably at least 250 amino acids, more preferably at least 300 amino acids, more preferably at least 400 amino acids, more preferably at least 500 amino acids, more preferably at least 600 amino acids, more preferably at least 700 amino acids, more preferably at least 800 amino acids that are homologous to a cockroach allergen as provided herein, i.e. SEQ ID NOs: 9-16. The term “homologous” as it refers herein to an amino acid sequence means that the amino acid sequence is at least 70%, more preferably 75%, more preferably 80%, more preferably 85%, more preferably 90%, more preferably 95%, more preferably 98%, or most preferably 100% identical to a known amino acid sequence (for example SEQ ID NOs: 9-16).

As used herein the term “polynucleotide including a sequence encoding a cockroach allergen or a fragment thereof” refers to a polynucleotide that includes a nucleotide sequence that encodes a cockroach allergen or a fragment of a cockroach allergen as defined herein. It is understood that there are many different nucleotide sequences that could encode a single polypeptide sequence based on normal base paring and codon usage relationships. As such, the term refers to a polynucleotide that includes any nucleic acid sequence that would encode a cockroach allergen or fragment thereof. In certain preferred embodiments, the polynucleotide including a “polynucleotide including a sequence encoding a cockroach allergen” of the invention includes a nucleotide sequence that is homologous to a sequence shown in SEQ ID NOs: 1-8. Preferably a “polynucleotide including a sequence encoding a cockroach allergen” includes a contiguous segment of at least 50 nucleotides; more preferably at least 100 nucleotides; more preferably at least 200 nucleotides; more preferably at least 300 nucleotides; more preferably at least 450 nucleotides; more preferably at least 600 nucleotides; more preferably at least 800 nucleotides; more preferably at least 1,000 nucleotides; more preferably at least 1,500 nucleotides; more preferably at least 2,000 nucleotides that are homologous to a sequence shown in SEQ ID NOs: 1-8. The term “homologous” as it refers herein to an nucleotide sequence means that the nucleotide sequence is at least 70%, more preferably 75%, more preferably 80%, more preferably 85%, more preferably 90%, more preferably 95%, more preferably 98%, or most preferably 100% identical to a known nucleotide sequence (for example sequence SEQ ID NOs: 1-8). It is understood that “polynucleotide including a sequence encoding a cockroach allergen” can contain additional nucleotides, other than the nucleotides forming a sequence that encode a cockroach allergen.

As used herein, the term “lipofection reagent” refers to a substance used to incorporate genetic material into a cell by means of liposomes. Examples of lipofection reagents include lipofectin, lipofectamine, cationic lipids and neutral co-lipids.

As used herein, the term “about” means in quantitative terms plus or minus 10%.

Other features and advantages of the invention will be apparent from the following description of the preferred embodiments and from the claims.

### DETAILED DESCRIPTION OF THE INVENTION

The invention relates to compositions and methods for managing cockroach allergies in mammals. In particular aspects, the invention relates to the administration to a mammal of nucleic acids that encode a cockroach allergen. The compositions are prepared and administered in such a manner that the cockroach allergen coding sequence is expressed in the mammal to which the composition is administered. These compositions include expression systems, delivery systems, and certain cockroach allergens coding sequences.

Allergic diseases have an immune response that deviates toward a T-helper type 2 (Th2) profile and away from the T-helper type 1 (Th1) profile. The Th2 profile is characterized by increased levels of interleukin (IL), such as IL-4, IL-5, IL-13, and the production of antigen specific IgE. IL-4 is important in IgE synthesis and development of the Th2 response, and IL-5 in eosinophil survival. Immunotherapy results in reversal of this imbalance, with increases in Th1 cytokines, IFN-γ and IL-12, which in turn inhibit the Th2 response. At the same time that genetic vaccination work is burgeoning, so is the work on the low affinity IgG receptor, FCγRIIB, which when occupied, inhibits the IgE-mediated response on mast cells and basophils (Daeron, et al., J Clin Invest. 95(2): 577-85 (1995)).

Allergic asthma can cause inflammatory disease of the bronchi and it is well established that a variety of cells including mast cells, eosinophils and lymphocytes play a role in this process. After an inhalation challenge, the inflammatory cells migrate from the peripheral blood to the site of inflammation in the bronchial mucosa and brochaoalveolar fluid shows dominant Th2-type cytokines. Our histological studies showed that the cockroach allergen vaccination induced the reduction of infiltration of inflammatory cells in lung tissues. The vaccine had an effect on the reduction of the inflammatory cells in lung tissue and genetic immunization affects not only humoral immune responses but also cellular responses.

New therapies designed for attenuation of allergic disease include soluble anti-IgE (Busse, et al., J Allergy Clin. Immunol. 108(2):184-90 (2001)), DNA vaccination with cDNA that codes for a targeted allergen (Homer, et al., J Allergy Clin. Immunol. 106(2):349-56 (2000)), and traditional or peptide immunotherapy combined with immunostimulatory DNA sequences (ISS-ODN) (Homer, et al., J Allergy Clin. Immunol. 106(2):349-56 (2000); and Jilek, et al., J. Immunol. 166(5): 3612-21 (2001)), either in solution, or covalently linked together. All show some promise of ameliorating allergic disease (Homer, et al., J Allergy Clin. Immunol. 106(2):349-56 (2000)), and traditional or peptide immunotherapy combined with immunostimulatory DNA sequences (ISS-ODN) (Homer, et al., J Allergy Clin. Immunol. 106(2):349-56 (2000); Jilek, et al., J Immunol. 166(5): 3612-21 (2001); Homer, et al., Immunol. Rev. 179:102-18 (2001); Nguyen, et al., Allergy 56 Suppl. 67:127-30 (2001); and Adel-Patient, et al., Int. Arch. Allergy Immunol. 2001; 126 (1): 59-67 (2001)).

Vaccination with cDNA requires fewer injections and has a quicker build-up phase. The risk of adverse reactions to immunotherapy may also be reduced. Plasmid DNA and its gene expression have been noted to be long lasting (Wolff, et al., Hum. Mol. Genet. 1: 363-69 (1992)) and immune responses in primates and rodents have been documented to last for more than one year following DNA vaccination (Donnelly, et al., J Immunol. Meth. 176: 145-152 (1994); and Raz, et al., Pro. Natl. Acad. Sci. 91: 9519-9523 (1994)). It does not appear that plasmid DNA is incorporated into the host genome, but remains as an episome (Tang, et al., Nature 356: 152-154 (1992)). The discovery that naked DNA and RNA is taken up and transiently expressed by muscle cells in vivo has increased interest in using non-viral vehicles for genetic delivery. See Wolff et al., Science 247: 1465-1468 (1990); Acsadi et al., Nature 352: 815-818 (1991). Although naked DNA and RNA can be taken up by mammalian cells, the efficiency of transfection is increased tremendously if the DNA or RNA is complexed in liposomes (Chen, et al., Gene Therapy 7(19): 1698-705 (2000)).

Administering a polynucleotide to a mammal in vivo, such that a cockroach allergen or fragment thereof is expressed in the mammal, can be achieved using any of many methods known in the art for mammalian gene expression. For example such methods for administering expressible polynucleotides to mammals including expression systems and delivery systems can be found in U.S. Pat. Nos. 6,875,748, 5,763,270, 5,580,859, 6,040,295, and 6,034,072.

Polynucleotide constructs described herein include nucleotide sequences encoding a cockroach allergen or fragment thereof. The polynucleotide is administered such that the polynucleotide is incorporated into cells and expresses a detectable amount of a prophylactically or therapeutically effective amount of a desired cockroach allergen or fragment thereof. Exemplary cockroach species are the German cockroach, Blatella germanica and the American cockroach, Periplaneta Americana. Exemplary cockroach allergens suitable for use in the invention include Bla g 1 (GB Accession No. AF072219), Bla g 2 (GB Accession No. U28863), Bla g 4 (GB Accession No. U40767), Bla g 5 (GB Accession No. U92412), Bla g 7 (GB Accession No. AF260897), Per a 1 (GB Accession No. U69957), Per a 3 (GB Accession No. L40818), and Per a 7 (GB Accession No. AF106961). SEQ ID NO:1 is the naturally occurring nucleotide sequence of Bla g 1. SEQ ID NO: 9 is the naturally occurring peptide sequence of Bla g 1. SEQ ID NO:2 is the naturally occurring nucleotide sequence of Bla g 2. SEQ ID NO: 10 is the naturally occurring peptide sequence of Bla g 2. SEQ ID NO:3 is the naturally occurring nucleotide sequence of Bla g 4. SEQ ID NO: 11 is the naturally occurring peptide sequence of Bla g 4. SEQ ID NO:4 is the naturally occurring nucleotide sequence of Bla g 5. SEQ ID NO: 12 is the naturally occurring peptide sequence of Bla g 5. SEQ ID NO:5 is the naturally occurring nucleotide sequence of Bla g 7. SEQ ID NO:13 is the naturally occurring peptide sequence of Bla g 7. SEQ ID NO:6 is the naturally occurring nucleotide sequence of Per a 1. SEQ ID NO: 14 is the naturally occurring peptide sequence of Per a 1. SEQ ID NO:7 is the naturally occurring nucleotide sequence of Per a 3. SEQ ID NO: 15 is the naturally occurring peptide sequence of Per a 7. SEQ ID NO:8 is the naturally occurring nucleotide sequence of Per a 1. SEQ ID NO: 16 is the naturally occurring peptide sequence of Per a 7.

The polynucleotides of the invention may be administered such that a combination two or more different cockroach allergens or fragments thereof are expressed in a mammal. This may be achieved either by administering two or more different polynucleotides that include a sequence encoding different cockroach allergens or fragments thereof, and/or by administering a polynucleotide that includes two or more sequences encoding different cockroach allergens or fragments thereof. As such, in certain preferred embodiments of the invention, the compositions and methods are designed for the administration of two or more; three or more; four or more; five or more; six or more different cockroach allergens or fragments thereof to a mammal. In particularly preferred embodiments the combination includes a cockroach allergen with an amino acid sequence homologous to Bla g 1 or a fragment thereof and a cockroach allergen with an amino acid sequence homologous to Bla g 2 or a fragment thereof.

Expression Systems

Non-viral administration of nucleic acid in vivo has been accomplished by a variety of methods. These include lipofectin/iposome fusion: Proc. Natl. Acad. Sci. 84: 7413-7417 (1993); polylysine condensation with and without adenovirus enhancement: Human Gene Therapy 3: 147-154 (1992); and transferrin:transferrin receptor delivery of nucleic acid to cells: Proc. Natl. Acad. Sci. 87: 3410-3414 (1990). The use of a specific composition consisting of polyacrylic acid has been disclosed in WO 94/24983. Naked DNA has been administered as disclosed in WO90/11092.

Thus, in one aspect, the invention provides a plasmid for expression of cockroach allergen or fragment thereof which includes an expression cassette, which can also be referred to as a transcription unit. When a plasmid of the invention is placed in an environment suitable for gene expression, the transcriptional unit will thus express the polynucleotide including a sequence encoding a cockroach allergen or fragment thereof. The transcription unit includes a transcriptional control sequence, which is transcriptionally linked with a cockroach allergen coding sequence. Transcriptional control sequence may include promoter/enhancer sequences such as cytomegalovirus (CMV) promoter/enhancer sequences. However, those skilled in the art will recognize that a variety of other promoter sequences suitable for expression in eukaryotic cells are known and can similarly be used in the constructs of this invention. The level of expression of the gene product will depend on the associated promoter and the presence and activation of an associated enhancer element. In certain embodiments, a sequence encoding a cockroach allergen or fragment thereof of the invention can be cloned into an expression plasmid which contains the regulatory elements for transcription, translation, RNA stability and replication (i.e. including a transcriptional control sequence). Such expression plasmids are well known in the art and one of ordinary skill would be capable of designing an appropriate expression construct with a polynucleotide including a sequence encoding a cockroach allergen or fragment thereof in such a manner that the cockroach allergen is expressible. There are numerous examples of suitable expression plasmids into which a polynucleotide including a sequence encoding a cockroach allergen or fragment thereof could be cloned such as pCI-neo and pcDNA3.1.

Large quantities of a bacterial host harboring a plasmid for expression of cockroach allergen or fragment thereof may be fermented and the plasmid may be purified for subsequent use. Current human clinical trials using plasmids utilize this approach. Recombinant DNA Advisory Committee Data Management Report, Human Gene Therapy 6: 535-548 (1994).

The purpose of the plasmid is to generally be used in human gene therapy for the efficient delivery of nucleic acid sequences to and expression of therapeutic genes (i.e. cockroach allergens) in a cell or tissue of a mammal. In particular, the purpose of the plasmid is to achieve high copy number, avoid potential causes of plasmid instability and provide a means for plasmid selection. As for expression, the nucleic acid cassette contains the necessary elements for expression of the nucleic acid within the cassette. Expression includes the efficient transcription of an inserted gene, nucleic acid sequence, or nucleic acid cassette with the plasmid. Expression products may be proteins, polypeptides or RNA. The nucleic acid sequence can be contained in a nucleic acid cassette. Expression of the nucleic acid can be continuous or regulated.

As an initial step in the process of ultimately obtaining expression of a product encoded by a nucleic acid, is to effect the uptake of the nucleic acid by cells. Uptake of nucleic acid by cells is dependent on a number of factors, one of which is the length of time during which a nucleic acid is in proximity to a cellular surface. For instance, after intramuscular (I.M.) administration of plasmid DNA in buffer, a marked reduction in gene expression is observed if the muscle is massaged, presumably due to DNA leakage out of the muscle either directly or via lymphatic vessels (Human Gene Therapy 4:151-159 (1993)). Accordingly, it would be desirable to formulate nucleic acids with compounds which would retard the rate at which nucleic acids diffuse or are carried away from a site at which cellular uptake of the nucleic acid is desired. Further, these compounds would be suitable for administration to an organism by means such as injection while maintaining or regaining the physical characteristics necessary to increase cellular uptake of nucleic acids.

Pharmaceutical Compositions

A composition comprising a polynucleotide including a sequence encoding a cockroach allergen or fragment thereof can be administered as a pharmaceutical composition wherein the invention compound is formulated with a pharmaceutically acceptable carrier as is well known in the art. Techniques for formulation and administration may be found, for example, in “Remington's Pharmaceutical Sciences,” (18th ed., Mack Publishing Co., Easton, Pa., 1990). Accordingly, the invention compounds may be used in the manufacture of a medicament. It is understood that a pharmaceutically acceptable carrier, or a pharmaceutical composition, or any substance suitable for administration to a mammal should be manufactured and stored in accordance with standards of local regulations. For example many governments have guidelines or rules that regulate various aspects of the manufacture and handling of compositions which are for administration into mammals and/or humans such as sanitation, process validation, equipment and document traceability, and personnel qualification. Preferably, a pharmaceutical composition or a pharmaceutically acceptable carrier of the invention is suitable for administration to a human and pharmaceutically complies with GMP (Good Manufacturing Practices) regulations set forth by the United States Food and Drug Administration for such a purpose.

Pharmaceutical compositions of the invention compounds may be formulated as solutions or lyophilized powders for parenteral administration. Powders may be reconstituted by addition of a suitable diluent or other pharmaceutically acceptable carrier prior to use. Powders also may be sprayed in dry form. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, compositions comprising a polynucleotide including a sequence encoding a cockroach allergen or fragment thereof may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. For aqueous compositions used in vivo, the use of sterile pyrogen-free water is preferred. Such formulations will contain an effective amount of a polynucleotide together with a suitable amount of an aqueous solution in order to prepare pharmaceutically acceptable compositions suitable for administration to a mammal, preferably a human. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. For rectal administration, the invention compounds may be combined with excipients such as cocoa butter, glycerin, gelatin or polyethylene glycols and molded into a suppository.

Administration of pharmaceutically acceptable salts of the polynucleotides described herein is included within the scope of the invention. Such salts may be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. For a helpful discussion of pharmaceutical salts, see S. M. Berge et al., Journal of Pharmaceutical Sciences 66: 1-19 (1977).

The invention also provides a pharmaceutical product for use in supplying a cockroach allergen polynucleotide including a sequence encoding a cockroach allergen or fragment thereof to a mammal. The pharmaceutical product may comprise a pharmaceutically effective amount of a polynucleotide including a sequence encoding a cockroach allergen or fragment thereof, a container enclosing the carrier and the polynucleotide in a sterile fashion, and/or means associated with the container for permitting transfer of the polynucleotide from the container to the interstitial space of a tissue, whereby cells of the tissue can take up and express the polynucleotide. The means for permitting such transfer can include a conventional septum that can be penetrated, e.g., by a needle. Alternatively, when the container is a syringe, the means may be considered to comprise the plunger of the syringe or a needle attached to the syringe. Containers used in the invention may have at least 1, preferably at least 5 or 10, and more preferably at least 50 or 100 micrograms of polynucleotide, to provide one or more unit dosages. For many applications, the container will have at least 500 micrograms or 1 milligram, and often will contain at least 50 or 100 milligrams of polynucleotide.

The invention also includes a pharmaceutical product, comprising a polynucleotide including a sequence encoding a cockroach allergen or fragment thereof, in physiologically acceptable administrable form, in a container, and a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert.

Polynucleotides including a sequence encoding a cockroach allergen or fragment thereof for injection, a preferred route of delivery, may be prepared in unit dosage form in ampules, or in multidose containers. The polynucleotides may be present in such forms as suspensions, solutions, or emulsions in oily or preferably aqueous vehicles. Alternatively, the polynucleotide salt may be in lyophilized form for reconstitution, at the time of delivery, with a suitable vehicle, such as sterile pyrogen-free water. Both liquid as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Nonionic materials, such as sugars, are preferred for adjusting tonicity, and sucrose is particularly preferred. Any of these forms may further comprise suitable formulatory agents, such as starch or sugar, glycerol or saline. The compositions per unit dosage, whether liquid or solid, may contain from 0.1% to 99% of polynucleotide material.

The units dosage ampules or multidose containers, in which the polynucleotides are packaged prior to use, may comprise an hermetically sealed container enclosing an amount of polynucleotide or solution containing a polynucleotide suitable for a pharmaceutically effective dose thereof, or multiples of an effective dose. The polynucleotide is packaged as a sterile formulation, and the hermetically sealed container is designed to preserve sterility of the formulation until use.

The container in which the polynucleotide including a sequence encoding a cockroach allergen or fragment thereof is packaged is labeled, and the label bears a notice in the form prescribed by a governmental agency, for example the Food and Drug Administration, which notice is reflective of approval by the agency under Federal law, of the manufacture, use, or sale of the polynucleotide material therein for human administration.

Federal law requires that the use of pharmaceutical agents in the therapy of humans be approved by an agency of the Federal government. Responsibility for enforcement is the responsibility of the Food and Drug Administration, which issues appropriate regulations for securing such approval, detailed in 21 U.S.C. § 301-392. Regulation for biologic material, comprising products made from the tissues of animals is provided under 42 U.S.C. § 262. Similar approval is required by most foreign countries. Regulations vary from country to country, but individual procedures are well known to those in the art.

The dosage to be administered depends to a large extent on the condition and size of the subject being treated as well as the frequency of treatment and the route of administration. Regimens for continuing therapy, including dose and frequency may be guided by the initial response and clinical judgment. The parenteral route of injection into the interstitial space of tissues is preferred, although other parenteral routes, such as inhalation of an aerosol formulation, may be required in specific administration, as for example to the mucous membranes of the nose, throat, bronchial tissues or lungs. The following is an example of vaccination schedules for humans at various age ranges; for individuals six to 35 months old, four doses of 0.5 mg of a vaccine comprising a polynucleotide encoding a sequence of a cockroach allergen or fragment thereof may be administered intramuscularly in the antereolateral aspect of the thigh or in the deltoid if muscle mass is sufficient at 0 months, 3 months, 6 months, and 12 months; for individuals three to eight years old, four doses of 1 mg of a vaccine comprising a polynucleotide encoding a sequence of a cockroach allergen or fragment thereof may be administered intramuscularly in the antereolateral aspect of the thigh or in the deltoid if muscle mass is sufficient at 0 months, 3 months, 6 months, and 12 months; for individuals 9 years and older, four doses of 3 mg of vaccine comprising a polynucleotide encoding a sequence of a cockroach allergen or fragment thereof may be administered intramuscularly in the deltoid at 0 months, 3 months, 6 months, and 12 months.

As such, the invention provides a pharmaceutical product, comprising a polynucleotide including a sequence encoding a cockroach allergen or fragment thereof, in solution in a physiologically acceptable injectable carrier and suitable for introduction interstitially into a tissue to cause cells of the tissue to express a cockroach allergen or fragment thereof, a container enclosing the solution, and a notice associated with the container in form prescribed by a governmental agency regulating the manufacture, use, or sale of pharmaceuticals, which notice is reflective of approval by the agency of manufacture, use, or sale of the solution of polynucleotide for human administration.

Administration

In any of the methods disclosed herein, it is preferred that the composition comprising polynucleotide including a sequence encoding a cockroach allergen or fragment thereof be delivered to a mammal. More preferably, the mammal is a human. Administration of the compositions of the invention according to any of the above methods can be accomplished according to any of various methods known in the art. For example, U.S. Pat. No. 5,676,954 discloses injection of genetic material, complexed with cationic lipid carriers, into mice. Also, U.S. Pat. Nos. 5,589,466, 5,693,622, 5,580,859, 5,703,055, and PCT international patent application PCT/US94/06069 (WO 94/29469), provide methods for delivering compositions comprising naked DNA or DNA-cationic lipid complexes to vertebrates.

In preferred embodiments, the compound comprising a polynucleotide including a sequence encoding a cockroach allergen or fragment thereof can be administered parenterally, intravascularly, intravenously, intraarterially, intramuscularly, intradermally, intravascularly, intracranially, subcutaneously, or the like. The compound can be introduced into muscle, skin, brain, lung, lymph node, liver or spleen tissue. The compound can also be introduced into the blood. Administration can also be orally, nasally, rectally, transdermally or inhalationally via an aerosol. The composition may be administered as a bolus, or slowly infused.

The polynucleotide including a sequence encoding a cockroach allergen or fragment thereof may be delivered to the interstitial space of tissues of the animal body, including those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts.

In vivo, muscle cells are particularly competent in their ability to take up and express a polynucleotide. This ability may be due to the singular tissue architecture of muscle, comprising multinucleated cells, sarcoplasmic reticulum, and transverse tubular system. Polynucleotides may enter the muscle through the transverse tubular system, which contains extra cellular fluid and extends deep into the muscle cell. It is also possible that the polynucleotides enter damaged muscle cells which then recover.

Muscle is also advantageously used as a site for the delivery and expression of polynucleotides in a number of therapeutic applications because animals have a proportionately large muscle mass which is conveniently accessed by direct injection through the skin; for this reason, a comparatively large dose of polynucleotides can be deposited in muscle by multiple injections, and repetitive injections, to extend therapy over long periods of time, are easily performed and can be carried out safely and without special skill or devices.

Tissues other than those of muscle may also be advantageously used as injection sites to produce cockroach allergens of the invention. One such condition is the use of a polynucleotide to provide a polypeptide which to be effective must be present in association with cells of a specific type; for example, the cell surface receptors of liver cells associated with cholesterol homeostasis (Brown and Goldstein, Science 232:34-47 (1986)). In this application, and in many others, such as those in which an enzyme or hormone is the gene product, it is not necessary to achieve high levels of expression in order to effect a valuable therapeutic result.

In certain embodiments, the polynucleotide including a sequence encoding a cockroach allergen or fragment thereof is introduced into tissues using an injectable carrier alone. The carrier preferably is isotonic, hypotonic, or weakly hypertonic, and has a relatively low ionic strength, such as provided by a sucrose solution. The preparation may further advantageously comprise a source of a cytokine which is incorporated into liposomes in the form of a polypeptide or as a polynucleotide.

Compounds comprising a polynucleotide including a sequence encoding a cockroach allergen or fragment thereof of the invention may be formulated to include other medically useful drugs or biological agents. The compounds also may be administered in conjunction with the administration of other drugs or biological agents useful for the disease or condition that the invention compounds are directed (see e.g., U.S. Pat. No. 6,413,955 for active ingredients useful for osteoporosis).

Compounds comprising a polynucleotide including a sequence encoding a cockroach allergen or fragment thereof of the invention may also be introduced into tissues or cells by a “gene gun.” DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

As employed herein, the phrase “an effective amount” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990.

Adjuvants

For delivery of a polynucleotide including a sequence encoding a cockroach allergen or fragment thereof to a mammalian system, it is usually preferable to utilize a delivery system. Such a system can provide multiple benefits, notably providing stabilization to protect the integrity of the DNA, as well as assisting in cellular uptake.

In addition, as illustrated by an exemplary delivery system described herein, the non-DNA components of the formulation can contribute to an immune system enhancement or activation. As a result, components of a delivery system can be selected in conjunction with a particular gene product to enhance or minimize the immuno-stimulatory effect.

Immunostimulatory effects are also described for certain nucleotide sequences. For example, Sato et al., Science 273: 352-354 (1996) describes the effects of vaccination with dsDNA having certain CpG containing sequences on the production of interferon-γ, interferon-β, and interleukin-12.

The polynucleotide including a sequence encoding a cockroach allergen or fragment thereof may be formulated with an adjuvant to enhance the resulting immune response. As well known in the art, adjuvants such as local anesthetics, preservatives, buffering agents, lubricants, wetting agents, colorants, flavorings, fillers and diluents may suitably be included in any of the methods and compositions of the invention. As used herein, the term “adjuvant” means a chemical that enhances the immune response to a vaccine. An adjuvant is distinguished from a carrier protein in that the adjuvant is not chemically coupled to the immunogen (i.e. polypeptide) or the antigen. Adjuvants are well known in the art and include, for example, mineral emulsions (U.S. Pat. No. 4,608,251) such as Freund's complete (emulsion of mineral oil, water, and mycobacterial extracts) or Freund's incomplete adjuvant (emulsion of water and oil only), (Freund, Adv. Tuberc. Res. 7: 130 (1956) (available from Calbiochem), mineral gels, e.g. aluminum salts, especially aluminum hydroxide or ALLOHYDROGEL (approved for use in humans by the U.S. Food and Drug Administration); surface active substances such as lysolecithin, polyanions, peptides, BCG (Bacillus Calmette-Guerin); muramyl dipeptide (MDP) and its analogs such as [Thr¹]-MDP (Byers and Allison, Vaccine 5: 223 (1987)), monophosphoryl lipid A (Johnson et al., Rev. Infect. Dis. 9:S512 (1987)), and the like.

Transfection Reagents

Compositions comprising a polynucleotide including a sequence encoding a cockroach allergen or fragment thereof of the invention can also include one or more transfection facilitating materials that facilitate delivery of polynucleotides to the interior of a cell, and/or to a desired location within a cell. Many such transfection facilitating materials are commercially available, for example Lipofectin, Lipofectamine, Lipofectamine 2000, Optifect, SuperFect. Examples of transfection facilitating materials include, but are not limited to lipids, preferably cationic lipids; inorganic materials such as calcium phosphate, and metal (e.g., gold or tungsten) particles (e.g., “powder” type delivery solutions); peptides, including cationic peptides, targeting peptides for selective delivery to certain cells or intracellular organelles such as the nucleus or nucleolus, and amphipathic peptides, i.e. helix forming or pore forming peptides; basic proteins, such as histones; asialoproteins; viral proteins (e.g., Sendai virus coat protein); pore-forming proteins; and polymers, including dendrimers, star-polymers, “homogenous” poly-amino acids (e.g., poly-lysine, poly-arginine), “heterogeneous” poly-amino acids (e.g., mixtures of lysine & glycine), co-polymers, polyvinylpyrrolidinone (PVP), and polyethylene glycol (PEG). Furthermore, those auxiliary agents of the invention which facilitate and enhance the entry of a polynucleotide into vertebrate cells in vivo, may also be considered “transfection facilitating materials.”

Lipofection facilitated transfection is well known in the art as described, for example, in U.S. Pat. Nos. 6,034,072, 6,040,295 and 6,710,035. Certain embodiments of the invention may include lipids as a transfection facilitating material, including cationic lipids (e.g., DOTMA, DMRIE, DOSPA, DC-Chol, GAP-DLRIE), basic lipids (e.g., steryl amine), neutral lipids (e.g., cholesterol), anionic lipids (e.g., phosphatidyl serine), and zwitterionic lipids (e.g., DOPE, DOPC). Preferably, the cationic lipid is mixed with one or more co-lipids. For purposes of definition, the term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films.

Delivery can also be through use of DNA transporters. DNA transporter refers to a molecule which binds to DNA vectors and is capable of being taken up by epidermal cells. DNA transporters contain a molecular complex capable of noncovalently binding to DNA and efficiently transporting the DNA through the cell membrane. A DNA transporter system can consist of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine. A first element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor. A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone. A third element is capable of binding to both the DNA vector and to elements which induce episomal lysis. Examples include inactivated virus particles such as adenovirus, peptides related to influenza virus hemagglutinin, or the GALA peptide.

In certain embodiments, naked polynucleotide materials, methods, and delivery systems are used, such as those described in U.S. Pat. Nos. 6,040,295, 5,763,270, and 5,580,859, according to the methods of the invention and include a polynucleotide including a sequence encoding a cockroach allergen or fragment thereof. These polynucleotides are naked in the sense that they are free from any delivery vehicle that can act to facilitate entry into the cell or any material which promotes transfection, such as liposomal formulations, charged lipids such as lipofectin or precipitating agents such as CaPO₄.

Viral vectors can also be used for transfection of a mammalian cell and introducing a polynucleotide including a sequence encoding a cockroach allergen or fragment thereof into the genome. In an indirect method, viral vectors, carrying new genetic information, are used to infect target cells removed from the body, and these cells are then re-implanted. Direct in vivo gene transfer into postnatal animals has been reported for formulations of DNA encapsulated in liposomes and DNA encapsulated in proteoliposomes containing viral envelope receptor proteins (Nicolau et al., Proc. Natl. Acad Sci USA 80:1068-1072 (1983); Kaneda et al., Science 243:375-378 (1989); Mannino et al., Biotechniques 6:682-690 (1988).

In a certain embodiments the viral vector is preferably a retroviral vector. Retroviral vectors are gene transfer plasmids wherein the heterologous nucleic acid resides between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764).

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, which provide a description of methods for efficiently introducing nucleic acids into human cells using such retroviral vectors. Other retroviral vectors include, for example, mouse mammary tumor virus vectors (e.g., Shackleford et al., Proc. Natl. Acad. Sci. U.S.A. 85:9655-9659 (1998)), lentiviruses, and the like. An exemplary viral vector is plentilox-IRES-GFP.

### EXAMPLE 1

### Manipulation of Target Gene: Preparation of Target Gene from pBluescript for Insertion into pCI-Neo

Cockroaches are homogenized, and total RNA is extracted by thoroughly grinding the cockroaches, washing the grindings twice with cold phosphate-buffered saline (PBS), centrifuging the grindings at 3,000×g for 10 min, and suspending the grindings in guanidine-phenol solution. First-strand cDNA is synthesized by incubating 5 μg of total RNA with 2.5 U of Moloney murine leukemia virus reverse transcriptase and oligo(dT)16 at 42° C. for 1 h in a 20 μl reaction volume and stored at −20° C. until further use.

PCR amplification is carried out for 35 cycles with the following conditions: 94° C. for 45s, 50° C. for 30s, and 68° C. for 2 min. The amplification products are purified by agarose gel electrophoresis and subcloned into the Xho1/BamH1 restriction site of the cloning vector pBluescriptSK+/−. The resulting plasmids carrying the subcloned PCR products are identified by restriction endonucleases (Xho1/BamH1) digestion, and the inserted sequences is confirmed by DNA sequencing. The verified sequences are cleaved from the pBluescriptSK+/−backbone by restriction endonucleases (Xho1/Xba1) digestion specific for the incorporated 5′ sites of the PCR products and subsequently ligated to the DNA vaccine vector pCI-neo, resulting in the conclusive DNA vaccine constructs. The inserted sequences are verified by DNA sequencing. For DNA vaccination experiments, the DNA vaccines encoding unique cockroach antigens are purified with the EndoFree plasmid purification kit (available from Qiagen).

### Manipulation of Target Gene: Preparation of Target Gene from pBluescript for Insertion into pcDNA3.1

Cockroaches are homogenized, and total RNA is extracted by thoroughly grinding the cockroaches, washing the grindings twice with cold phosphate-buffered saline (PBS), centrifuging the grindings at 3,000×g for 10 min, and suspending the grindings in guanidine-phenol solution. First-strand cDNA is synthesized by incubating 5 μg of total RNA with 2.5 U of Moloney murine leukemia virus reverse transcriptase and oligo(dT)16 at 42° C. for 1 hr. in a 20 μl reaction volume and stored at −20° C. until further use.

PCR amplification is carried out for 35 cycles with the following conditions: 94° C. for 45s, 50° C. for 30s, and 68° C. for 2 min. The amplification products are purified by agarose gel electrophoresis and subcloned into the Sma1/Kpn1 restriction site of the cloning vector pBluescriptSK+/−. The resulting plasmids carrying the subcloned PCR products are identified by restriction endonucleases (Sma1/Kpn1) digestion, and the inserted sequences are confirmed by DNA sequencing. The verified sequences are cleaved from the pBluescriptSK+/− backbone by restriction endonucleases (BamH1/Kpn1) digestion specific for the incorporated 5′ sites of the PCR products and subsequently ligated to the DNA vaccine vector pcDNA3.1 (available through Invitrogen), resulting in the conclusive DNA vaccine constructs. The inserted sequences are verified by DNA sequencing. For DNA vaccination experiments, the DNA vaccines encoding unique cockroach antigens were purified with the EndoFree plasmid purification kit.

### Manipulation of Target Gene: Preparation of Target Gene from pBluescript for Insertion into Plentilox-IRES-GFP

Cockroaches are homogenized, and total RNA is extracted by thoroughly grinding the cockroaches, washing the grindings twice with cold phosphate-buffered saline (PBS), centrifuging the grindings at 3,000×g for 10 min, and suspending the grindings in guanidine-phenol solution. First-strand cDNA is synthesized by incubating 5 μg of total RNA with 2.5 U of Moloney murine leukemia virus reverse transcriptase and oligo(dT) 16 at 42° C. for 1 h in a 20 μl reaction volume and stored at −20° C. until further use.

PCR amplification is carried out for 35 cycles with the following conditions: 94° C. for 45s, 50° C. for 30s, and 68° C. for 2 min. The amplification products are purified by agarose gel electrophoresis and subcloned into the Xho1/Sam1 restriction site of the cloning vector pBluescriptSK+/−. The resulting plasmids carrying the subcloned PCR products are identified by restriction endonucleases (Xho1/Sam1) digestion, and the inserted sequences are confirmed by DNA sequencing. The verified sequences are cleaved from the pBluescriptSK+/− backbone by restriction endonucleases (Xho1/BamH1) digestion specific for the incorporated 5′ sites of the PCR products and subsequently ligated to the DNA vaccine vector plentilox-IRES-GFP, resulting in the conclusive DNA vaccine constructs. The inserted sequences are verified by DNA sequencing. For DNA vaccination experiments, the DNA vaccines encoding unique cockroach antigens are purified with the EndoFree plasmid purification kit.

### EXAMPLE 2

### Plasmid Construct

Total mRNA is isolated from German cockroaches (Blatella germanica) as described in Example 1. First strand cDNA is generated from total mRNA and by reverse transcriptase PCR (RT-PCR). The cDNA is used for PCR using Taq polymerase with primers specific for Bla g 2. These primers cover the mature excreted region of Bla g 2 and include restriction enzyme sites Xho1 and BamH1 for cloning. The amplified PCR products and pBluescript plasmids are subject to endo-nuclease digestion by Xho1 and BamH1. The amplified products are ligated and cloned into pBluescript plasmids. The Bla g 2 clones in pBluescript are then amplified using PCR to create a 1.1 kb Bla g 2 fragment for subcloning. The 1.1 kb Bla g 2 PCR products and mammalian expression vector pCI-neo plasmids (available from Promega) are subjected to endonuclease digestion by Xba1 and Xho1. The 1.1 kb Bla g 2 products are ligated and cloned into pCI-neo plasmids.

### EXAMPLE 3

### In Vitro Testing

The plasmid constructs from Example 2 are then subjected to in-vitro testing. Each plasmid construct is prepared using EndoFree Plasmid Maxi Protocol (available from Qiagen). The plasmid construct is transfected into human lung epithelial cell line with lipofection reagent for 24 hours. Total RNA is extracted and cDNA is generated by RT-PCR. Primers are designed to amplify the 1.1 kb fragment of Bla g 2 from the cDNA. Cells lines transfected with the Bla g 2 plasmid construct expressed Bla g 2 mRNA.

In addition, protein expression of the transfected human lung epithelial cells is measured by ELISA. Cells lines transfected with the plasmid construct expressed Bla g 2 protein.

### EXAMPLE 4

### Immunization

A. Immunization

Two groups of C57BL6 mice are injected with 50 μg of plasmid DNA intramuscularly three times at weekly intervals. The control group receives blank plasmid and the experimental group receives pCI-neo+Bla g 2 plasmid. The plasmid DNA is emulsified with an equal volume of complete Freund's adjuvant (CFA) for immunization. The first group is vaccinated with pCI-neo blank vector PBS (control mice) and the second group is vaccinated with pCI-neo+Bla g 2 plasmid construct in PBS (experimental mice). Two weeks after the last immunization, the mice are sensitized three times at two week intervals by an initraperitoneal (IP) dose of 2 μg of Bla g 2 protein, 300 ng of pertussis toxin, and 1 mg of aluminum. Four weeks after the last sensitization, the mice are nasally challenged with 10 μg of Bla g 2 protein. Four days later the mice are sacrificed.

B. Cytokine Assays

Serum is assayed for cytokines IFN-γ, IL-13 and IL-5. The lymph nodes from the sacrificed mice are removed and washed with PBS buffer and mRNA is prepared. First strand cDNA is generated from 1 μg of total RNA by using murine leukemia virus reverse transcriptase and random hexonucleotide primers with the Perkin Elmer Gene Amp RNA PCR kit (Perkin Elmer). Primers are designed to amplify IFN-γ, IL-13 and IL-5.

A negative control reaction is run with each sample to verify that no PCR bands appeared in the absence of template. Amplification conditions are as follows: 45s at 94° C. for denaturation, 45s at 67° C. for annealing, and 1 minute at 72° C. for elongation for 30 cycles. The products are run on an agarose gel, dried on Whatman 3M paper, and exposed to Kodak XAR film. In each electrophoresis run, intra- and inter-gel staining homogeneity is confirmed by staining intensity of molecular markers at both ends of the gels. In general, amplification kinetics are monitored for each PCR run by examining aliquots of the products on the gel. PCR products are compared during cycles which amplification has not yet reached saturation.

Mice administered with pCI-neo+Bla g 2 had increased IFN-γ, IL-13 and IL-5 mRNA expression.

C. Histological Study

To examine the vaccination effect on cellular response, lung tissue is used for histological examination. Mice are anesthetized by intramuscular injection with a 0.2 ml of ketalar (35 mg/ml), rompun (0.6% ml) and atropine (0.1 mg/ml). The vascular bed of the lungs is perfused with 0.01M PBS followed by 4% paraformaldehyde 0.1M PBS. Whole lungs are taken out and stored in 4% paraformaldehyde for 24 hrs at 4° C. After fixation, the tissue is dehydrated and embedded in parafilm. Frozen sections cut at 6 μm in thickness are stained by hematoxylin and eosin. The sections are observed using light microscopy.

The lung tissue of mice administered with pCI-neo+Bla g 2 had less infiltration of inflammatory cells around bronchioli, blood vessels and interstitium than the control group. The result suggests that the Bla g 2 DNA vaccine has an effect on cellular response and provided protection against subsequent allergic challenges.

### EXAMPLE 5

### Lipofection Effect

A. Immunization

Six groups of C57BL6 mice are injected with 50 μg of plasmid DNA intramuscularly three times at weekly intervals. The plasmid DNA is emulsified with an equal volume of complete Freund's adjuvant (CFA) for immunization. Mice are immunized either by intramuscular injection (IM) or intranasally. Within each administration method are three groups: 1) pCI-neo+Bla g 2 with lipofectin (25 μg/μl), 2) pCI-neo with lipofectin (25 μg/μl), and 3) lipofectin only (25 μg/μl). Two weeks after the last immunization, the mice are sensitized three times at two week intervals by an intraperitoneal (IP) dose of 2 μg of Bla g 2 protein, 300 ng of pertussis toxin, and 1 mg of aluminum. Four weeks after the last sensitization, the mice are nasally challenged with 10 μg of Bla g 2 protein. Four days later the mice are sacrificed.

B. Tritiated Thymidine Uptake

After immunization, lymph node cells are removed aseptically and single-cell suspension was prepared. Cells were cultured in 200 μl RPMI1640 supplemented with 10% fetal calf serum (available from Hyclone Laboratories), 1 mmol/L sodium pyruvate, 100 μg/ml penicillin, 100 μg/ml streptomycin, 2 mmol/L glutamine, 5×10-5 mol/L 2-mercaptoethanol, 20 mmol/L HEPES (pH 7.4), and 50× nonessential amino acids. After culturing for 72 hours, 1 μCi of [3H] thymidine labeled antigens specific for IFN-γ, IL-2, IL-13 or IL-5 (available from Du Pont) is added to each well. Eighteen hours later, cells are harvested and measured by liquid scintillation counting. Values are expressed in counts per minute as follows: counts per minute with antigen-counts per minute without antigen. Each sample is run in triplicate.

Tritiated thymidine uptake was significantly increased in Bla g 2 treated cells from all animals than the control groups which did not receive Bla g 2. There were no significant differences among the Bla g 2 groups.

Splenocyte Cytokine Assay

Splenocytes are assayed for cytokines IFN-γ, IL-2, IL-4, IL-5 and IL-13. The splenocytes from sacrificed mice are removed and washed with PBS buffer and mRNA is prepared. First strand cDNA was generated from 1 μg of total RNA by using murine leukemia virus reverse transcriptase and random hexonucleotide primers with the Perkin Elmer Gene Amp RNA PCR kit (Perkin Elmer). Primers are designed to amplify IFN-γ, IL-2, IL-4, IL-5 and IL-13.

A negative control reaction is run with each sample to verify that no PCR bands appeared in the absence of template. Amplification conditions are as follows: 45s at 94° C. for denaturation, 45s at 67° C. for annealing, and 1 minute at 72° C. for elongation for 30 cycles. The products are run on an agarose gel, dried on Whatman 3M paper, and exposed to Kodak XAR film. In each electrophoresis run, intra- and inter-gel staining homogeneity is confirmed by staining intensity of molecular markers at both ends of the gels. In general, amplification kinetics are monitored for each PCR run by examining aliquots of the products on the gel. PCR products are compared during cycles which amplification had not yet reached saturation.

IFN-γ expression increased significantly in the Bla g 2 treated cells from all animals than the control groups. Intramuscular Bla g 2 administration yielded greater uptake than intranasal Bla g 2 administration. IL-13 mRNA expression was significantly less than the Bla g 2 immunized mice compared to the liposome only group and slightly less than the plasmid plus liposome group. IL-2 also had increased mRNA expression. Both IL-4 and IL-5 had a slight decrease in mRNA expression than the groups without Bla g 2.

C. Histological and Immunology Studies

To examine whether the Bla g 2 vaccine affected cellular response, lung tissue is stained by histological and immunohistochemical methods. After the mice are sacrificed, lung, trachea, and nose tissue are taken for histological examination. Tissues are fixed with periodat-lysine-paraformaldehyde solution for 24 h at 4° C. Frozen sections are cut at 4 to 6 μm in thickness and rehydrated and rinsed in cold PBS. The endogenous pseudoperoxide is blocked with methanol containing 0.5% hydrogen peroxide for 20 min at room temperature. The sections are then treated with 10% normal goat serum in PBS to reduce nonspecific binding. Biotin conjugated rat anti-mouse CD8 or CD4 monoclonal antibodies (available from Pharmingen) diluted 1:200 in PBS containing 0.5% bovine serum albumin, are applied to the sections and incubated overnight at 4° C. The sections are rinsed with PBS and incubated with streptavidin-biotin peroxidase complexes (Vecastain Elite ABC Kit, Vector Laboratories) for 30 min at room temperature and rinsed with PBS. The reaction is developed with 0.02% 3,3′-diaminobenzidine in 0.05 M Tris buffer (pH 7.6) with 0.005% hydrogen peroxide for 7 min. The sections are then dehydrated, cleared in xylene, and mounted.

Immunization with Bla g 2 decreased inflammation of lung tissue after a Bla g 2 allergen challenge. CD8 cells increased while CD4 cells decreased in Bla g 2 immunized groups. Whereas in groups without Bla g 2 administration, there were more CD4 cells than CD8 cells. This suggests that the Bla g 2 vaccine affects cellular response and CD8 T cells were capable of protection against subsequent allergy challenges. T lymphocytes have been suggested to play a key role in orchestrating the interaction of the cells since they can release various cytokines which can attract, primer, and activate other cell types. A successful outcome of immunotherapy has been associated with the development of suppressor T cells, which can down-regulate an allergic response. Functionally distinct subsets of CD8 T cells reportedly play a regulatory role in IgE production. Cystolic proteins are generally presented to CD8 T cells by major histocompatibility complex (MHC) class I molecules which are expressed on virtually all somatic cells. It is suggested that endogenous production of allergenic protein might be a useful means to induce regulatory CD8 T cells capable of conferring protection against subsequent allergenic challenges.

### EXAMPLE 6

### Bla g 1 Assays

A. Sera Assay

BALB/c mice are vaccinated by injection with 100 μg of blank pcDNA3.1 (control group) or pcDNA3.1+Bla g 1 (experimental group) three times at weekly intervals into the muscle of the mice. After 3 weeks, mice are actively sensitized twice with 100 μg of cockroach extract and also nasally challenged with 10 μg of cockroach extract six times at weekly intervals. Sera is collected six times. Sera from seven mice of each group are pooled and used for determination of cockroach specific IgE antibodies produced.

The experimental group had decreased IgE production compared to the control group.

B. Histopathologic Examination

To examine whether genetic vaccination affects cellular response to the lungs, lungs are stained at the end of the experiment by histological and immunohistochemical methods known in the art. Lungs from control and experimental groups of mice were removed on day 45 after immunization.

Lungs from the control group showed much more infiltration by inflammatory cells in the submucosa of large and medium sized airways than lungs of the experimental group. Also, eosinophils were detected in the lungs of the control mice. Eosinophils and many other infiltrated cells were observed in other tissues from the control group. Decreased infiltration of the lungs was observed in tissues from animals from the experimental group than in the control group.

C. Immunohistochemical Examination

Lungs from control and experimental mice are removed on day 45 after immunization and are stained for CD8⁺ cells.

More CD8⁺ T cells were observed in tissues from the experimental mice compared to control mice. The immunohistochemical stain for CD4⁺ T cells and CD8⁺ T cells showed that more CD8⁺ T cells infiltrated the submucosa and mucosa of lungs from the experimental group as compared with lungs from the control group. The stain for CD4⁺ T cells showed no difference between the two groups. So the results suggest that genetic vaccination also affects the cellular response and that the CD8⁺ T cells of the experimental group were capable of protecting against a subsequent allergenic challenge.

D. Cytokine Gene Expression by Antigen Stimulation in vivo.

To determine whether Th1 or Th2 cytokines are involved in genetic vaccination, T cells are harvested from lymph nodes of mice and stimulated with cockroach extract in vivo. Total RAN is extracted using Trizol reagent. RT-PCR reactions are done using cDNA with different primers specific for β-actin, IL-2, IL-4, IL-5 and IFN-γ.

Higher levels of mRNA expression of IFN-γ and IL-2 were detected in the experimental group compared with expression in the control group. There was greater mRNA expression of IL-4 and IL-5 in the control group than the experimental group. These data indicate that vaccination with Bla g 1 genes induce Th1 cytokine (IFN-γ) gene expression in lymph nodes.

Two weeks after immunization of BALB/c mice with cockroach extract and DNA vaccination, IFN-γ and IL-2 mRNA expression were detected in the experimental group, but were not detected in the control group. In contrast, IL-4 and IL-5 mRNA expression levels were detected in the control group. The results suggest that the genetic immunization with the plasmid DNA encoding Bla g 1 also induces a Th2 to TH1 cytokine shift.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

That which is claimed is:

1. A method of treating or prophylaxis of an allergen in a mammal, said method comprising administering to said mammal a polynucleotide comprising a sequence encoding a cockroach allergen or a fragment thereof.

2. The method according to claim 1, wherein said cockroach allergen is homologous to a cockroach allergen selected from the group consisting of Bla g 1, Bla g 2, Bla g 4, Bla g 5, Bla g 6,Per a 1, Per a 3, and Per a 7.

3. The method according to claim 1, wherein said polynucleotide comprises a sequence encoding a fragment of a cockroach allergen with an amino acid sequence homologous to a sequence of a cockroach allergen selected from the group consisting of Bla g 1 (SEQ ID NO:9), Bla g 2 (SEQ ID NO:10), Bla g 4 (SEQ ID NO:11), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO: 16).

4. The method according to claim 3, wherein said fragment of a cockroach allergen comprises an amino acid sequence that is at least 70% homologous to a sequence of a cockroach allergen selected from the group consisting of Bla g 1 (SEQ ID NO:9), Bla g 2 (SEQ ID NO:10), Bla g 4 (SEQ ID NO:11), Bla g 5 (SEQ ID NO: 12), Bla g 6 (SEQ ID NO:13), Per a 1 (SEQ ID NO:14), Per a 3 (SEQ ID NO:15), and Per a 7 (SEQ ID NO:16).

5. The method according to claim 4, wherein said fragment of a cockroach allergen comprises at least 25 amino acids homologous to a sequence of a cockroach allergen selected from the group consisting of Bla g 1 (SEQ ID NO:9), Bla g 2 (SEQ ID NO: 10), Bla g 4 (SEQ ID NO:11), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO:16).

6. The method according to claim 1, wherein said polynucleotide comprises a nucleotide sequence homologous to a nucleotide sequence selected from the group consisting of Bla g I (SEQ ID NO:1), Bla g 2 (SEQ ID NO:2), Bla g 4 (SEQ ID NO:3), Bla g 5 (SEQ ID NO:4), Bla g 6 (SEQ ID NO:5), Per a I (SEQ ID NO:6), Per a 3 (SEQ ID NO:7), and Per a 7 (SEQ ID NO:8).

7. The method according to claim 1, wherein said polynucleotide comprises a nucleotide sequence of at least 100 contiguous nucleotides that are homologous to a nucleotide sequence selected from the group consisting of Bla g 1 (SEQ ID NO:1), Bla g 2 (SEQ ID NO:2), Bla g 4 (SEQ ID NO:3), Bla g 5 (SEQ ID NO:4), Bla g 6 (SEQ ID NO:5), Per a 1 (SEQ ID NO:6), Per a 3 (SEQ ID NO:7), and Per a 7 (SEQ ID NO:8).

8. The method according to claim 1, wherein said polynucleotide comprises a nucleotide sequence of at least 100 contiguous nucleotides that are at least 70% identical to a nucleotide sequence selected from the group consisting of Bla g 1 (SEQ ID NO:1), Bla g 2 (SEQ ID NO:2), Bla g 4 (SEQ ID NO:3), Bla g 5 (SEQ ID NO:4), Bla g 6 (SEQ ID NO:5), Per a 1 (SEQ ID NO:6), Per a 3 (SEQ ID NO:7), and Per a 7 (SEQ ID NO:8).

9. The method according to claim 1, wherein said polynucleotide comprises circular DNA comprising an origin of replication (OR1), promoter, and multiple cloning site (MCS).

10. The method according to claim 1, wherein said polynucleotide is a plasmid comprising promoter/enhancer transcriptionally linked to said sequence encoding a cockroach allergen.

11. The method according to claim 1, wherein said promoter is suitable for expression in eukaryotic cells.

12. The method according to claim 1, wherein said method comprises administering said polynucleotide with a transfection facilitating material.

13. The method according to claim 12, wherein said transfection facilitating material comprises a lipid.

14. The method according to claim 1, wherein said administering comprises one or methods selected from the group consisting of injection, inhalation, and gene gun.

15. The method according to claim 1, wherein said administering comprises one or more methods selected from the group consisting essentially of intravenous injection, intramuscular injection, intraperitoneal injection, and subcutaneous injection.

16. The method according to claim 1, wherein said mammal is a human.

17. A method for generating an immune response in a mammal, said method comprising administering to said mammal a polynucleotide comprising a sequence encoding a cockroach allergen or a fragment thereof.

18. A composition comprising a polynucleotide comprising a sequence encoding a cockroach allergen or fragment thereof and a pharmaceutically acceptable carrier.

19. The composition according to claim 18, wherein said polynucleotide comprises a sequence encoding a fragment of a cockroach allergen with an amino acid sequence homologous to a sequence of a cockroach allergen selected from the group consisting of Bla g 1 (SEQ ID NO:9), Bla g 2 (SEQ ID NO:10), Bla g 4 (SEQ ID NO:11), Bla g 5 (SEQ ID NO:12), Bla g 6 (SEQ ID NO:13), Per a 1 (SEQ ID NO:14), Per a 3 (SEQ ID NO:15), and Per a 7 (SEQ ID NO:16).

20. The composition according to claim 18, wherein said polynucleotide is a plasmid comprising promoter/enhancer transcriptionally linked to said sequence encoding a cockroach allergen.

21. The composition according to claim 18, wherein said promoter is suitable for expression in eukaryotic cells.

22. The composition according to claim 18, wherein said composition further comprises a transfection facilitating material comprising a lipid.

23. The composition according to claim 18, wherein said composition is suitable for injection into a human or for inhalation by a human.

24. A method of preparing a composition for expression of a cockroach allergen or fragment thereof in a mammal, said method comprising preparing a polynucleotide comprising a promoter enhancer transcriptionally linked to a sequence encoding a cockroach allergen or a fragment thereof; preparing a transfection facilitating material; and combining said transfection facilitating material with said polynucleotide.

25. A method for detecting sensitivity to a cockroach allergen in an individual, said method comprising administering a polynucleotide comprising a sequence encoding a cockroach allergen or a fragment thereof to the individual and determining an allergic response.


SEQUENCE LISTING (16 sequences)

SEQ ID NO: 1 - DNA, 1429 bp, Blattella germanica
aatcttctagaaaaactgcgtgaaaagggagttgatgtggacaaaattattgaactcatcagagctctttttggactgaccttaaatgccaaggcctcccgcaacctccaagatgacctccaggatttcttggccctcattcctgttgatcaaatcattgccattgccacagactaccttgctaatgatgctgaagtacaggctgccgttgcatacctccaatctgatgaatttgagactattgttgtagctcttgatgcactcccagaattgcagaacttcctcaacttcttagaggccaatggactcaatgcaattgacttcctcaatggcatccatgatcttcttggaattcctcacattccagtctcaggacgcaaatatcacatccgcagaggagttggaatcactggtctcattgacgatgtcctcgccatccttcccattgaagacctgaaggccctctttaacgagaaattggaaactagcccagatttccttgctctgtacaatgccatccgttcccctgaattccagagcattgtccagacattgaatgccatgccagaataccagaatctcctgcaaaaacttcgtgagaagggagttgatgtggacaaaatcattgaactgatcagggccctatttggactgactctaaatggcaaagcttcccgcaacctccaagatgacctccaagatttcttggcactcattcctgtagaccaaattattgccattgccacagactaccttgctaatgatgctgaagtacaggctgccgttgcatacctccaatctgatgaatttgagactatcgttgtaactcttgatgcactcccagaattgcagaatttcctcaacttcttggaggccaatggactcaacgcaattgacttcctcaacggcatccatgatcttcttggaattcctcacattccagtctcaggacgcaaatatcacatccgcagaggagttggaatcactggtctcattgatgatgtcctcgccatccttccccttgatgacctgaaagccctctttaacgagaaattggaaactagcccagatttccttgctctgtacaatgccatcaaatcccctgaatttcagagtattgttcaaacactgaatgccatgccagaatatcagaatcttctcgaaaagctgcgtgaaaagggagtcgacgtagacaaaatcatcgaactcatcagggccctgtttggactcactcactagtgagaactcaagactgatatgtttaagtgccaaacttcatttctgataataatcagaatgttaacattgttcattttttgaacacttgtattatattttgattgttccacgaataaatacttcaaaataaaaaaaaaaaaaaaaaaaaaaaaaaaaaaaaaaaaaaaaaaaaaaaaaaaaaaaaaaaaaa

SEQ ID NO: 2 - DNA, 1317 bp, Blattella germanica
aaatgattggcctaaagctagtgacagttctctttgcggttgctaccataacacatgcagctgagcttcaacgtgttccattgtacaaattggtgcacgttttcattaacactcaatacgctggtataaccaagattggaaaccagaacttcctaacagtattcgatagcacctcatgcaatgtagtcgttgccagtcaagaatgcgttggtggagcttgtgtatgtccaaatctacaaaaatatgagaaacttaaaccgaagtatatctctgatgggaatgtacaggtgaaattcttcgacactggtagcgcagttggtagaggcattgaagattcccttacgatttctaacctcacgacatctcaacaagacattgtccttgccgatgaactcagtcaagaagtctgcattctatctgctgacgtagttgtaggaatagcagccccaggatgccctaatgcactgaaaggaaaaactgttctcgaaaactttgtcgaagaaaatcttattgcgcctgtcttttctattcatcatgctagatttcaagatggagaacatttcggagaaattattttcggaggttctgattggaaatacgttgatggtgaattcacttatgttccacttgtgggtgatgattcctggaagttcaggctggatggtgtgaaaataggtgacacaactgttgctccagcaggtacacaggccatcatcgacacaagcaaagctatcattgtcggacctaaagcctatgttaatccaatcaacgaagctattgggtgtgtagtggaaaagacaacaaccaggagaatatgcaagcttgactgcagcaagataccatctctccctgatgtcacatttgtgatcaatggcaggaatttcaacatcagctcacaatattacatccaacagaacgggaacttgtgctattccggcttccaaccatgcggtcactccgatcacttttttattggtgacttctttgttgatcattattattctgaattcaactgggagaacaagaccatgggattcggccgttcagtagaaagcgtctaagaatttcaacatcaagatggacttcagagattacttcggaatcactaataagacattcacgagacttacgaagaccactacagttttggatatgaatgatgacaaataactgaagacttttcattatatgacatggagaagatttttttaaagtcgcctattattacttttttcgcacacttttatgtatacagctactgatgtcttaaaataaactggaaatattttgaattttctaaaaaaaaaaaaaaaaaaa

SEQ ID NO: 3 - DNA, 601 bp, Blattella germanica
cgcagttttggcactatgtgcaacagatacattggcgaacgaagattgttttagacatgaatcattggttccaaaccttgattatgaaaggttcagaggttcgtggattattgcagccggcacttccgaagcgctcacccaatacaaatgctggatcgacaggttttcatatgacgatgcgttggtttctaagtatactgattcacaaggaaagaataggactactatcagaggacgaactaaatttgaaggcaacaagtttactatcgattataatgataaagggaaagcattttctgcgccatactctgttctagcaactgattacgaaaattacgcaattgtggaaggctgtcccgctgcagctaatggacatgtaatttatgttcaaatccgattttcggtgaggagatttcaccccaagctgggtgataaagaaatgatacagcactacactttggatcaggtgaatcaacacaagaaggctatagaagaagacttaaagcacttcaatttgaagtacgaggacttacactccacatgtcactaagtatgaaatgttcatatttattgtaggaaaataaaaccttctaatgaatta

SEQ ID NO: 4 - DNA, 1140 bp, Blattella germanica
cttataaactgacatactgtcccgtgaaggctctgggagagccaattcgcttccttctgtcttatggagagaaagattttgaagattatcgtttccaggagggagattggcctaatttgaaaccttccatgccatttggtaaaacaccagtgttggagattgatgggaagcaaacacaccagtctgttgccatttctcgctatcttggtaagcagtttggcctcagtggtaaggatgattgggagaacttggagatcgacatgatcgtcgacaccatctctgacttcagggctgccattgctaattaccattatgatgctgatgaaaattcaaagcagaagaaatgggaccctctcaagaaggaaaccattccttactacaccaaaaagtttgatgaagtggtgaaggctaacggaggataccttgctgctggaaagctgacatgggcagacttctacttcgttgccattctcgactatttgaatcacatggctaaagaagacctggtggccaatcaacccaatttgaaggctttgcgggagaaagtattgggtttgcctgctatcaaagcatgggtcgccaagcgtcctcctacagatctgtaagaaaaatgtgccatggcaaaaaaattcatgttgcatgtaacactgagatcataacgatgttctaaaagaaattttgttacgcataatgattttatgaaagtattttgttagcagctttgctctataataatcactagaccatatttaaaaggcaaaaacgaacattttcttcataaaaggcaaaaatagccaaaaaatacttttgtattaaaatattcattgacgctgattcttacatttaattcttcacaatttaagaattttttaacaatagtaattacgatcaacatttcagatctgtttagatatgattgcaaagcttgtttataatcagaaaatgacttcctaaaatcaacagcatatggcgcaaaatttttcgttctaaatttccagttttttaaatgtataatttttttggtaaacttttatttactagaaatttgatccagaagtagactgataatttcctttacttactttttggtattaaacaaagttggaaacaaaataattttgaaaaaaaaaaaaaaaaaaa

SEQ ID NO: 5 - DNA, 1115 bp, Blattella germanica
atggatgccatcaagaagaagatgcaggcgatgaagctggagaaggacaacgcgatggatcgcgcccttctctgcgaacagcaggcccgcgacgccaacatccgggccgagaaggctgaggaggaggcccgatccctgcagaagaagatccagcagattgagaatgatcttgatcagaccatggagcagttgatgcaagtcaacgccaagctggacgagaaggacaaggccctgcagaatgctgagagtgaggtcgctgccctcaaccgccgaatccaactgctggaggaggatcttgagaggtctgaggaacgtttggccacagccaccgccaagttggctgaggcttcccaggctgccgatgagtcagagcgagctcgtaagattcttgaatccaaaggcctggcagatgaagagcgtatggatgctttggagaaccagctgaaggaagccaggttcatggctgaggaagctgacaagaaatatgatgaggtcgcacgtaaattagcaatggttgaggccgacttggaaagagcagaagagcgtgccgagactggtgaatccaagattgtggagcttgaggaagaactgcgcgttgtcggcaacaacctgaagtcccttgaggtgtctgaagagaaggccaacctgcgtgaggaagagtacaagcaacagattaagactctgaataccaggctaaaggaggctgaagctcgtgctgagttcgctgaaagatccgtgcagaaattgcagaaggaggttgacaggcttgaggatgaattggtacacgagaaggagaagtacaagtacatttgtgacgatcttgatatgactttcaccgaacttattggcaactaaactactacaactgttctctggatcttgttcagttggaaccctgagttcacatagcagaccatggcagacaatgaactacttcttgcctcattgtaaaggtgctataataaagttggtgatattttaaaggttgcatttaaaaatcaccaaagtattttaaacccacagtgctatgtggactcactgtgtagtttggtcaacaacaatttcaaaagtgtaaagaaattaaaataaacatcttatttactaaaaaaaaaaaa

SEQ ID NO: 6 - DNA, 1432 bp, Periplaneta americana
atcagaggagcattagttaagatgaagcttccgattatgattttggcagtgttgggagttgcttttggcaagtccctccccaaccgcaacctccaggatgacctcaacgacttcctggcccttctgcccgtggacgaaataaccgccattgttatggactacctcgctaacgatgctgaagtacaagaggctgtggcttatttgcaaggagaagaattccacaagattgtttttacagttgaaggactacaggaattcgggaacttcgtccaattcttggaagaccacggccttgatgccgtaggttatatcaaccgactccactcagtatttggttgggatccctacgtaccatcatccaaacgcaagcatactcgcagaggcgttggtgtcgatggtctcatagacgacatcattgcaattcttcctattgacgatctgaaggctctcttccaggagaagctggaaactagtccagacttcaaagccttctacgatgctgtacggtctcctgaatttcagagcattgtgcagactctgaacgccatgccagagtaccaagaccttctgcagaaactgcgagacaaaggagtagatgttgatcactacatcgagctcatcagggcactcttcggcctcactcgagcggcacgcaatctgcaagatgacctgaacgacttcctggcactcattcccaccgatcagatactcgccatcgccatggactacctcgccaacgacgccgaagtacaagagctagtggcatacctccagtctgacgacttccacaaaattatcaataccattgaagcactccccgaattcgctaacttctacaatttcctcaaggggcacggtctcgatgtcgcggactacatcaacgagatccactccatcattggcttgccaccatttgtcccaccatctcgaagacacgctcgcagaggtgtaggaatcaatggactaattgatgatgtcatcgccattcttccagttgatgagttgaaggctctcttccaggagaaactggagactagccccgacttcaaggccctttatgatgctatacgatctccggaattccagagcataatttcaactctaaatgccatgccagagtaccaagaactactacagaatcttcgtgacaaagcagttgatgtggaccacttcatcgagttgatccgcagcttgttcggcctgccctgatactaacaaaacactttagatattttgtagaaaaatcgtgaccgaattgctatgttttgactattcagttctaatgccagaataactgatccatttgcaaaaatcatatgcattgctgtatgattatgtgtgtgatgacatttaaaataaacgtaacactcgcatgcattaaaaaaaaaaaaaaaaaaaaaaaaaaaaaaaaaaaaaaaaaaaaaaaaaaaaa

SEQ ID NO: 7 - DNA, 2418 bp, Periplaneta americana
gaccaccatgaagactgctttggtattcgctgcggttgtggccttcgtggctgcgcgcttccctgatcacaaggattacaaacagctcgccgacaaacaattcctcgccaagcagagggacgtgctgagactgttccacagagtccatcaacacaatatcctcaatgatcaggtcgaagtaggcatacctatgacatcgaagcaaacatcggcaactacagtacccccgagtggtgaagcagttcatggcgtacttcaagaagggcatgctcgtcctcgaggggagcccttctccgtaaactacgagaagcacagggagcaagccatcatgctgtacgacctgctctacttcgccaacgactatgacaccttctacaagaccgcctgctgggcacgtgatcgtgtgaatgaaggaatgttcatgtattccttcagcattgccgtcttccaccgtgacgacatgcagggtgtcatgctcccacccccgtatgaagtctatccctacctctttgtcgaccatgacgtcatccatatggctcagaaatactggatgaagaacgctggcagcggtgagcaccactcccacgtcattccagtgaacttcactctcagaacccaggatcacctcctggcctacttcacgtccgacgtgaacctcaacgccttcaacacgtactaccgctactactaccctagctggtacaacaccacactctacggacacaacatcgaccgccgtggtgagcagttctactatacctacaagcagatctatgctcgctacttcctggagaggctttccaacgatcttcccgatgtgtaccccttctactacagcaagcccgtcaagagtgcgtacaaccctaatctcaggtaccacaatggcgaagaaatgccggtgcgtcctagcaacatgtacgtcaccaacttcgacctgtactacatcgccgacatcaagaattacgaaaagagggttgaagatgccatcgacttcggttatgccttcgatgaacatatgaagccccactctttgtaccacgatgtccacggcatggagtaccttgcagatatgatcgagggtaacatggactctcctaacttctatttctacggttccatctaccatatgtaccactcaatgattggacacatcgtagatccataccacaagatggggctcgcacctagtctggaacatcccgagaccgttctgagggatcctgttttctatcaattgtggaagcgcgtggatcacctcttccagaaatacaagaacaggctgccccgttacactcacgatgagttggcattcgaaggtgtgaaagtggagaatgtggatgttggtaaactgtacacttacttcgagcagtacgatatgagcttggatatggccgtgtacgtcaacaacgtggatcagatctctaacgtggacgtccagctcgcagtacgtctcaaccacaagcctttcacctacaacattgaggtcagcagcgacaaggcacaggacgtgtacgtggccgtgttcttgggtcccaagtacgactacctgggccgcgaatacgacctgaacgatcgcagacactacttcgtagagatggatcgcttcccataccatgttggagctggaaagactgtgattgaacgcaactcccatgactcaaacatcatcgctcctgagagagacagctacaggacgttctacaagaaggtgcaagaggcttacgaaggaaagagccaatactatgtcgacaagggccacaactactgcggctacccagagaacctgctgatccccaagggcaagaagggaggccaggcctacaccttctacgtcatcgtaaccccttacgtcaagcaagacgaacacgacttcgagccttacaactacaaggccttcagctactgcggcgttggatccgagcgcaagtatcccgacaacaagcctctgggataccccttcgaccgcaagatctacagcaacgacttctacactcccaacatgtacttcaaggatgtgatcatcttccacaagaagtacgatgaggttggagtccaaggccattaagtgggcgatgcaataacagatttcaacttcacacatgatgaatggaaatgttttccttacttcgaaaacttaaacctataaacatgaaaaccaagacgtcagatctcaccatcagtaacgagtacaacaggtttctataattgaagtactgaatccatttcatgtaaataaattacattcaacaaccatttttcagccgttgaaatctgtaattatgtcaattctgctatggagaacggatattctgctgtactggagacaagtttttatactgagctttctgtaatgtgtacgtcaaagtatgatagagcaataaacaaacaatcttacaaactaaaaaaaaaaaaaaaaaa

SEQ ID NO: 8 - DNA, 1325 bp, Periplaneta americana
aattcggcacgaggagccgctgtacatccgtctgccagcccgtagaaagcaacaaaccaagccgccaccatggacgcgatcaagaagaagatgcaggcgatgaagctggagaaggacaacgcgatggaccgcgcccttctctgcgagcagcaggcccgcgacgccaaccttagggccgagaaggctgaggaggaggcgcgctccctgcagaagaagatccagcagattgagaacgatctggatcagaccatggagcagctgatgcaggtcaacgccaagctggacgagaaggacaaggccctccagaacgctgagagtgaagttgctgccctgaaccgccgtatccagctgctagaggaggacctcgagaggtctgaggaacgattggccactgccactgccaaactggccgaagcttcacaggctgccgatgaatccgagcgagcccgtaagattcttgaatccaagggcctggcagatgaagaacgcatggacgcactggagaaccagctgaaggaggccaggttcatggctgaagaagcagacaaaaaatatgatgaggttgcacgtaagttggccatggttgaggccgacttggagagagcagaggaacgtgccgagagtggtgaatccaaaattgtagagcttgaggaggagctccgtgttgtcggcaacaacctgaagtcccttgaggtgtctgaggagaaggccaacctgcgtgaagaagagtacaagcagcagattaagaccttgactaccaggctaaaggaggctgaggcccgtgctgagttcgctgagaggtccgtgcagaagctgcagaaggaggttgacaggcttgaggatgaattggtacacgagaaggagaagtacaagttcatttgtgatgatcttgatatgactttcaccgaacttattggcaactaaactactaccaactgttctgtggctcttgttcagttgcattgcctgggttcacatagtggggacagccccgacctccttcaaaggtgctaatattggtggagttttaatgaaactttaaaaattcaccataatatttataatctaacaacagactatgtggacccatcttgtagtttggtcaacaagaaaaaaaattcaagaagtgtaaaaatttaaaatatcttatttgctatgtttagaacttttatttggttggtataatttttttctctgttaattatgaactatgtttatattaatttgtcttatttcttaatttgggattatttatggtttttattaaaacaatgtataaaagattaaagaaagaaaattgatatgttaaaaaaaaaaaaaaaaaa

SEQ ID NO: 9 - protein, 412 aa, Blattella germanica
NLLEKLREKGVDVDKIIELIRALFGLTLNAKASRNLQDDLQDFLALIPVDQIIAIATDYLANDAEVQAAVAYLQSDEFETIVVALDALPELQNFLNFLEANGLNAIDFLNGIHDLLGIPHIPVSGRKYHIRRGVGITGLIDDVLAILPIEDLKALFNEKLETSPDFLALYNAIRSPEFQSIVQTLNAMPEYQNLLQKLREKGVDVDKIIELIRALFGLTLNGKASRNLQDDLQDFLALIPVDQIIAIATDYLANDAEVQAAVAYLQSDEFETIVVTLDALPELQNFLNFLEANGLNAIDFLNGIHDLLGIPHIPVSGRKYHIRRGVGITGLIDDVLAILPLDDLKALFNEKLETSPDFLALYNAIKSPEFQSIVQTLNAMPEYQNLLEKLREKGVDVDKIIELIRALFGLTH

SEQ ID NO: 10 - protein, 352 aa, Blattella germanica
MIGLKLVTVLFAVATITHAAELQRVPLYKLVHVFINTQYAGITKIGNQNFLTVFDSTSCNVVVASQECVGGACVCPNLQKYEKLKPKYISDGNVQVKFFDTGSAVGRGIEDSLTISNLTTSQQDIVLADELSQEVCILSADVVVGIAAPGCPNALKGKTVLENFVEENLIAPVFSIHHARFQDGEHFGEIIFGGSDWKYVDGEFTYVPLVGDDSWKFRLDGVKIGDTTVAPAGTQAIIDTSKAIIVGPKAYVNPINEAIGCVVEKTTTRRICKLDCSKIPSLPDVTFVINGRNFNISSQYYIQQNGNLCYSGFQPCGHSDHFFIGDFFVDHYYSEFNWENKTMGFGRSVESV

SEQ ID NO: 11 - protein, 182 aa, Blattella germanica
AVLALCATDTLANEDCFRHESLVPNLDYERFRGSWIIAAGTSEALTQYKCWIDRFSYDDALVSKYTDSQGKNRTTIRGRTKFEGNKFTIDYNDKGKAFSAPYSVLATDYENYAIVEGCPAAANGHVIYVQIRFSVRRFHPKLGDKEMIQHYTLDQVNQHKKAIEEDLKHFNLKYEDLHSTCH

SEQ ID NO: 12 - protein, 200 aa, Blattella germanica
YKLTYCPVKALGEPIRFLLSYGEKDFEDYRFQEGDWPNLKPSMPFGKTPVLEIDGKQTHQSVAISRYLGKQFGLSGKDDWENLEIDMIVDTISDFRAAIANYHYDADENSKQKKWDPLKKETIPYYTKKFDEVVKANGGYLAAGKLTWADFYFVAILDYLNHMAKEDLVANQPNLKALREKVLGLPAIKAWVAKRPPTDL

SEQ ID NO: 13 - protein, 284 aa, Blattella germanica
MDAIKKKMQAMKLEKDNAMDRALLCEQQARDANIRAEKAEEEARSLQKKIQQIENDLDQTMEQLMQVNAKLDEKDKALQNAESEVAALNRRIQLLEEDLERSEERLATATAKLAEASQAADESERARKILESKGLADEERMDALENQLKEARFMAEEADKKYDEVARKLAMVEADLERAEERAETGESKIVELEEELRVVGNNLKSLEVSEEKANLREEEYKQQIKTLNTRLKEAEARAEFAERSVQKLQKEVDRLEDELVHEKEKYKYICDDLDMTFTELIGN

SEQ ID NO: 14 - protein, 395 aa, Periplaneta americana
MKLPIMILAVLGVAFGKSLPNRNLQDDLNDFLALLPVDEITAIVMDYLANDAEVQEAVAYLQGEEFHKIVFTVEGLQEFGNFVQFLEDHGLDAVGYINRLHSVFGWDPYVPSSKRKHTRRGVGVDGLIDDIIAILPIDDLKALFQEKLETSPDFKAFYDAVRSPEFQSIVQTLNAMPEYQDLLQKLRDKGVDVDHYIELIRALFGLTRAARNLQDDLNDFLALIPTDQILAIAMDYLANDAEVQELVAYLQSDDFHKIINTIEALPEFANFYNFLKGHGLDVADYINEIHSIIGLPPFVPPSRRHARRGVGINGLIDDVIAILPVDELKALFQEKLETSPDFKALYDAIRSPEFQSIISTLNAMPEYQELLQNLRDKAVDVDHFIELIRSLFGLP

SEQ ID NO: 15 - protein, 685 aa, Periplaneta americana
MKTALVFAAVVAFVAARFPDHKDYKQLADKQFLAKQRDVLRLFHRVHQHNILNDQVEVGIPMTSKQTSATTVPPSGEAVHGVLQEGHARPRGEPFSVNYEKHREQAIMLYDLLYFANDYDTFYKTACWARDRVNEGMFMYSFSIAVFHRDDMQGVMLPPPYEVYPYLFVDHDVIHMAQKYWMKNAGSGEHHSHVIPVNFTLRTQDHLLAYFTSDVNLNAFNTYYRYYYPSWYNTTLYGHNIDRRGEQFYYTYKQIYARYFLERLSNDLPDVYPFYYSKPVKSAYNPNLRYHNGEEMPVRPSNMYVTNFDLYYIADIKNYEKRVEDAIDFGYAFDEHMKPHSLYHDVHGMEYLADMIEGNMDSPNFYFYGSIYHMYHSMIGHIVDPYHKMGLAPSLEHPETVLRDPVFYQLWKRVDHLFQKYKNRLPRYTHDELAFEGVKVENVDVGKLYTYFEQYDMSLDMAVYVNNVDQISNVDVQLAVRLNHKPFTYNIEVSSDKAQDVYVAVFLGPKYDYLGREYDLNDRRHYFVEMDRFPYHVGAGKTVIERNSHDSNIIAPERDSYRTFYKKVQEAYEGKSQYYVDKGHNYCGYPENLLIPKGKKGGQAYTFYVIVTPYVKQDEHDFEPYNYKAFSYCGVGSERKYPDNKPLGYPFDRKIYSNDFYTPNMYFKDVIIFHKKYDEVGVQGH

SEQ ID NO: 16 - protein, 284 aa, Periplaneta americana
MDAIKKKMQAMKLEKDNAMDRALLCEQQARDANLRAEKAEEEARSLQKKIQQIENDLDQTMEQLMQVNAKLDEKDKALQNAESEVAALNRRIQLLEEDLERSEERLATATAKLAEASQAADESERARKILESKGLADEERMDALENQLKEARFMAEEADKKYDEVARKLAMVEADLERAEERAESGESKIVELEEELRVVGNNLKSLEVSEEKANLREEEYKQQIKTLTTRLKEAEARAEFAERSVQKLQKEVDRLEDELVHEKEKYKFICDDLDMTFTELIGN